(19) United States
(12) Patent Application Publication
(10) Pub. No.: US 2010/0216225 A1
(43) Pub. Date: Aug. 26, 2010

(54) PORTABLE MICROORGANISM DETECTION UNIT

(75) Inventors: Rex Bryan Dyer, East Peoria, IL (US); Beth Susan Turnbull-Dyer, East Peoria, IL (US)

Correspondence Address: HUSCH BLACKWELL SANDERS LLP, 190 CARONDELET PLAZA, SUITE 600, ST. LOUIS, MO, 63105-3441, US

(73) Assignee: Ag-Defense Systems, Inc., Peoria, IL (US)

(21) Appl. No.: 12/392,683

(22) Filed: Feb. 25, 2009

(51) Int. Cl. C12M 1/36; C12M 1/34

(52) U.S. Cl. 435/286.6; 435/287.2

(57) ABSTRACT

The present invention is a portable device for detecting microorganisms in a food sample. The device includes a DNA extraction tube, a chip, and a portable control unit. The DNA extraction tube extracts DNA from the sample in an aqueous solution. The extraction tube includes successive chambers that are configured to mince, filter, and purify DNA in the form of an aqueous eluate. The chip includes reaction chambers disposed for a polymerase chain reaction to amplify a specific DNA fragment in the eluate for detection, and the portable control unit is configured to receive the chip and analyze the amplified DNA on the chip.

### BACKGROUND OF THE INVENTION

1. Field of the Invention

The present invention relates to microorganism detection devices and, more particularly, to a portable device for detecting pathogens on food and crops originating via unintentional or intentional adulteration. Unintentional adulteration of crops pertains to environmental pathogens normally present and that contaminate the food product during normal food processing procedures. Intentional adulteration of pathogens refers to a deliberate planting of pathogens on food or crops as would occur in an act of bioterrorism.

2. Related Art

The need to detect microorganisms in food products has increased in recent years. Specifically, food products may be contaminated with pathogens either intentionally or unintentionally. Often food products are tested for such microorganisms at the time that the food product is made. More often, the existence of such microorganisms does not become evident until after the food products have been sold at retail stores. Detecting a microorganism at such a late time generally results in a recall of vast amounts of food and/or a need to dispose of entire crops. Such recalls put an economic burden on food related businesses and pose a public health problem.

Most food manufacturers regularly analyze their food products. However, the detection systems in use today generally require the crop and/or food product to be tested at an isolated facility that is remote from the processing plant or the field. Accordingly, the testing is time consuming and costly. Moreover, during the time of testing, contaminated food products may make their way into general commerce only to be recalled at a later date.

U.S. Pat. Nos. 6,120,985, 6,111,096, and 6,274,726, each issued to Laugharn, Jr. et al. describe methods for cell lysis of biological materials for purifying DNA for downstream applications. The methods include exposing biological cells to elevated pressures in a pressure chamber. In one patent the cells are cooled to subzero temperatures prior to placing them in the chamber. The sample chamber includes a filter and a solid phase material. The filter operates to remove cell debris from the sample. The solid phase material includes any one of silica gel, glass, plastic, membrane, resins, hydroxyapatite or tethered specific binding molecules or metals that are used to bind to nucleic acids in the sample. After the nucleic acids in the sample bind to the solid phase, the pressure in the chamber is increased to release the nucleic acids from the solid phase. The cartridge or chip with the solid phase material also contains electrodes to move biomolecules towards the solid phase for binding or towards a waste or collection reservoir. Modulating the pressure at the solid phase changes the binding of the biomolecule on the solid phase material. However, known devices for isolating and purifying DNA do not allow for integrated point-of-use analysis with solid food matrices.

### SUMMARY OF THE INVENTION

Accordingly, it is desirable to provide a portable microorganism detection device that is capable of detecting microorganisms on food items, crops, or seeds in a point-of-use manner. Specifically, it is desirable to have a device that can be operated at the crop site or food processing plant to provide immediate analysis. It is also desirable to have a device that is capable of analyzing seeds prior to crop planting. Although the present application is written with respect to detecting microorganisms on food products, as will be appreciated by one of ordinary skill in the art, the present invention may be used to detect microorganisms on a vast array of products or environmental samples.

In one aspect, the present invention includes a portable device for detecting a microorganism in a food sample. The device includes a DNA extraction tube, a chip, and a portable control unit. The DNA extraction tube is configured to give a purified DNA eluate after extracting DNA from the sample. The tube includes an upper chamber having at least one mincing disk to mince the sample, a filter to filter particulate matter from the extract, and a lower chamber having a binding material configured to bind to the DNA of the extract. The chip is configured to retain the eluate for analysis. The chip includes reaction chambers disposed for an isothermal polymerase chain reaction to amplify the DNA in the eluate for detection. The portable control unit is configured to receive the chip and analyze the amplified DNA on the chip. In one embodiment, the device is configured to detect bioterrorism molecules and/or plant and food pathogens.

In another aspect, the present invention includes a DNA extraction tube for extracting DNA from a food sample. The tube includes an upper chamber and a lower chamber. The upper chamber is configured to receive the sample, and includes at least one mincing disk to mince the sample. The mincing disk is configured with serrated edges to facilitate mincing the sample. The lower chamber is configured to receive an extract from the upper chamber after a valve opens to let the extract passes through a filter for removing particulate matter. The lower chamber includes a binding material configured to bind to DNA from the extract. The lower chamber further includes an inlet for channeling fluids to the lower chamber and an outlet for channeling fluids from the lower chamber. In one embodiment, the extraction tube includes a plurality of consecutive mincing disks having incrementally smaller pore sizes.

In yet another aspect, the present invention includes a chip for analyzing DNA in a food sample. The chip includes reaction chambers disposed for an isothermal polymerase chain reaction to amplify the DNA for detection. The chip is also sized for insertion into a portable control unit that analyzes the DNA after amplification. In the exemplary embodiment, the chip further includes reagents in the reaction chambers for amplification of the DNA. The reagents include of lyophilized nucleotides, DNA polymerase, pathogen specific primers, buffers and salts, and/or fluorescent labeling molecules. The DNA is analyzed to detect food and plant pathogens and/or bioterrorism molecules.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a schematic view of a system for detecting microorganisms in a food sample.

FIG. 2 is a schematic view of a DNA extraction tube used with the system shown in FIG. 1.

FIG. 3 is a top view of a lysis disc used with the DNA extraction tube shown in FIG. 2.

FIG. 4 is a top view of a pore defined in the lysis disc shown in FIG. 3.

FIG. 5 is a schematic view of a chip used with the system shown in FIG. 1.

FIG. 6 is a schematic view of a portable control unit used with the system shown in FIG. 1.

FIG. 7 is a schematic view of the fluorescent detection system used with the system shown in FIG. 1.

### DETAILED DESCRIPTION OF THE PREFERRED EMBODIMENTS

The following description of the preferred embodiment(s) is merely exemplary in nature and is in no way intended to limit the invention, its application, or uses.

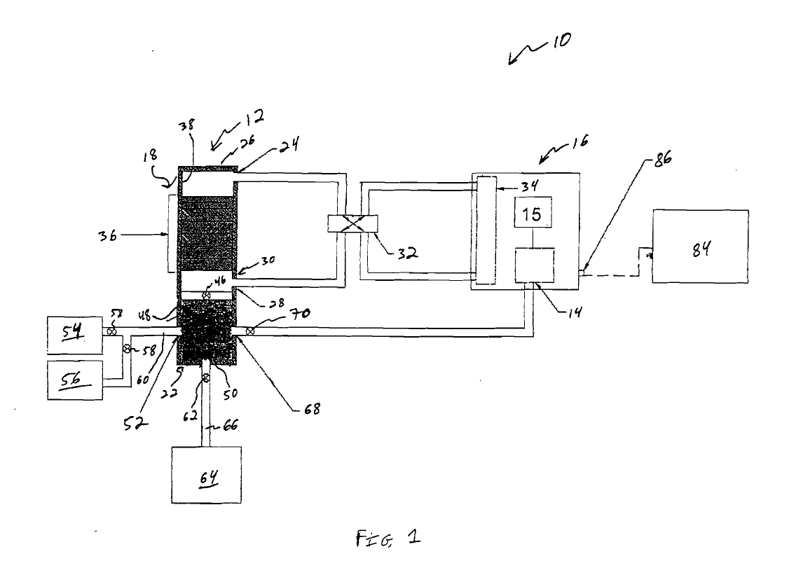

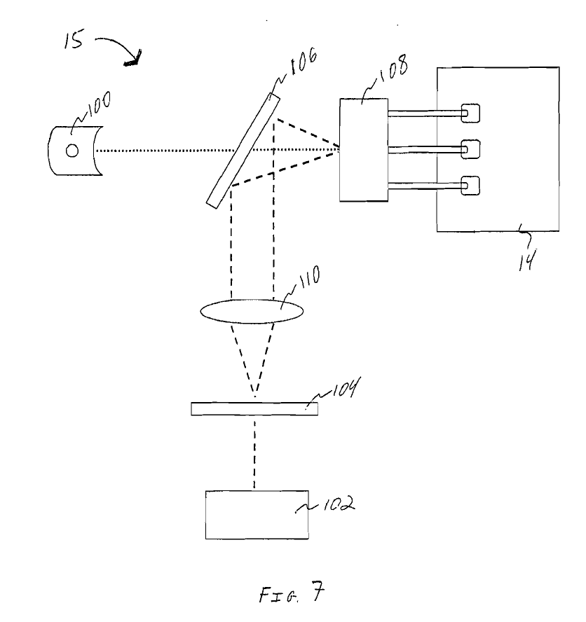

The present invention, as shown in FIGS. 1-7 is a portable device 10 for detecting microorganisms in a food sample. Specifically, in the exemplary embodiment, the device 10 is configured to detect plant and food pathogens and bioterrorism molecules in seeds, crops, and other food products. For example, the device 10 may be configured to detect pathogens such as soybean rust, wheat scab, corn ear rot, Salmonella, Listeria, Escherichia coli, and/or bioterrorism threats such as anthrax, bubonic plague, and/or yellow fever that are contained in food products such as meat, bottled water, fresh vegetables, dairy, seafood, and grains. The device 10 is portable so that the detection can take place at a point-of-use or, more specifically, at the location where the food is grown and/or produced. Accordingly, the food sample is not required to be taken to a location remote from the crop or processing facility to be analyzed.

As shown in FIG. 1, the device 10 includes a DNA extraction tube 12 for extracting and purifying a DNA eluate from the sample, a chip 14 configured to retain and react with the eluate to specifically amplify a portion of the DNA, and a portable control unit 16 configured to pressurize the DNA extraction tube 12. The control unit 16 is also configured to receive the chip 14 and analyze the amplified DNA. Although the present invention is described with respect to detecting microorganisms on food products, as will be appreciated by one of ordinary skill in the art, the present invention may be used to detect microorganisms on a vast array of products.

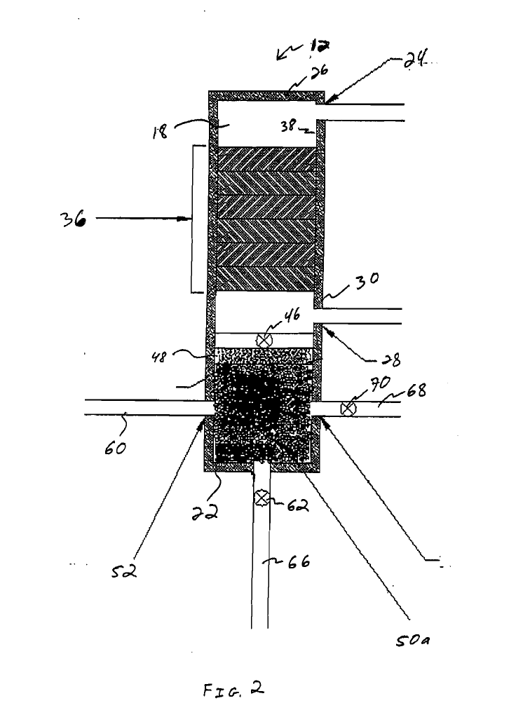

The DNA extraction tube 12, as shown in FIG. 2, includes two chambers separated by a filter. Specifically, an upper chamber 18, and a lower chamber 22. The food sample is placed in the upper chamber of the extraction tube 12 with a liquid extraction buffer. In the exemplary embodiment, the food sample is approximately 25 grams. The solution is then subject to varying pressures to extract the DNA. Specifically, the upper chamber 18 includes a pair of high pressure ports. A first high pressure port 24 is disposed at an upper end 26 of the upper chamber 18, and a second high pressure port 28 is disposed at a lower end 30 of the upper chamber 18. The high pressure ports are fluidly coupled to a two-way solenoid valve 32, which is coupled to a pressure module 34 in the control unit 16. The control unit 16 controls the pressure within the upper chamber 18 by alternating high pressure between the two high pressure ports 24, 28. More specifically, the solenoid valve 32 alternates the high pressure between the first high pressure port 24 and the second high pressure port 28, thereby causing the sample to move in alternating directions through the upper chamber 18. For example, when the high pressure is applied to the first high pressure port 24, the sample is forced downward through the upper chamber 18. Alternatively, when the high pressure is applied to the second high pressure port 28, the sample is forced upward through the upper chamber 18.

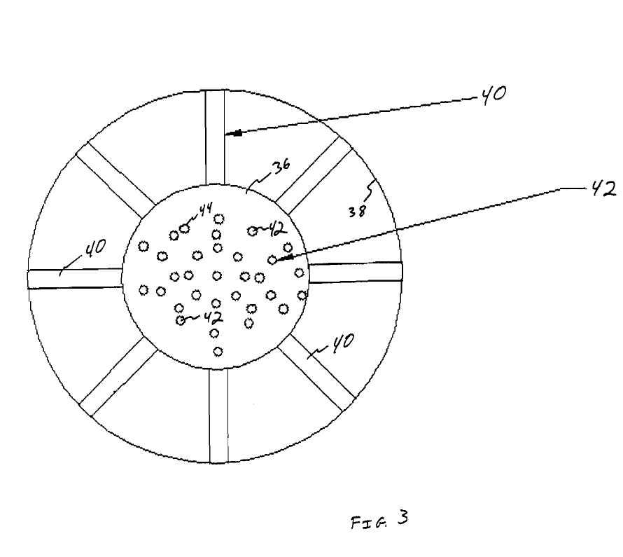

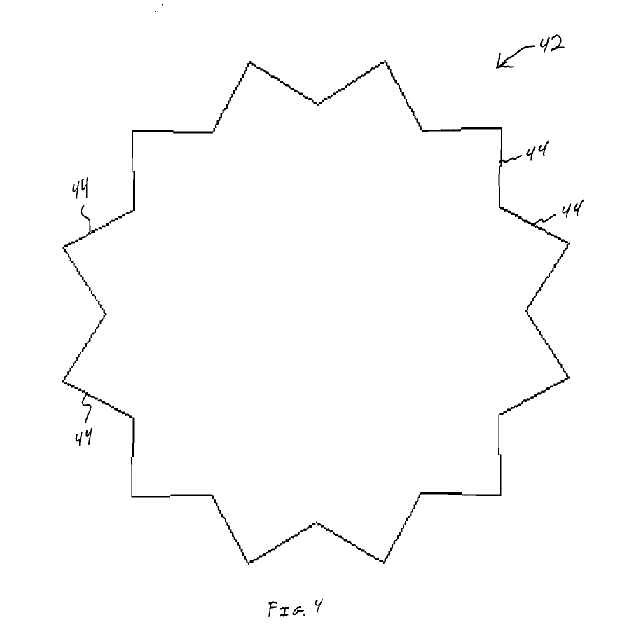

The upper chamber 18 includes at least one mincing disk 36, as shown in FIG. 3, positioned between the first high pressure port 24 and the second high pressure port 28. The mincing disk 36 is secured to the inner surfaces 38 of the DNA extraction tube 12 via a locking element 40 that secures the mincing disk 36 during the application of high pressure to the upper chamber 18. The mincing disk 36 further includes a plurality of pores 42 extending through the disk 36. As high pressure is applied to the upper chamber 18 in alternating directions, the sample is forced upward and downward through the mincing disk 36 to mince the sample so that the sample material ruptures, leading to the extraction of biomolecules into the extraction buffer, resulting in the formation of a lysed solution. In the exemplary embodiment, the pores 42 and/or the locking element 40 include serrated edges 44 configured to further mince the sample as it moves through the mincing disk 36. FIG. 4 illustrates one example of the serrated edges 44 of the pores 42. As will be appreciated by one of ordinary skill in the art, the serrated edges 44 are not limited to those shown in FIG. 4, but rather, the serrated edges 44 may any include various geometrical forms. Moreover, in one embodiment, as shown in FIG. 1, the upper chamber 18 includes a plurality of mincing disks 36 configured with incrementally smaller pore sizes so that the sample is minced into successively smaller pieces as it is forced downward through the disks 36.

In one embodiment, a premincing step is included that utilizes a stator/rotor positioned in the upper chamber 18. The rotating blade draws fluid and food into the stator and chops the food. The chopped food is forced out of holes in the stator to aid in further mincing. This premincing step occurs prior to forcing the food through the mincing disks 36 via pressure. In addition, in one embodiment, the pressure in the system is combined with methods such as ultrasonics. In a further embodiment, a hypotonic solution is used to separate the sample by flowing water into the cells of the sample causing them to swell and burst. In another embodiment, cycles of freeze/thawing leads to repetitive ice crystal formation which helps to break open the cell membranes of the sample.

The DNA extraction tube 12 includes a valve 46 positioned between the upper chamber 18 and the lower chamber 22 to seal the upper chamber 18 from the lower chamber 22. Accordingly, as high pressure is being applied to the upper chamber 18, the valve 46 is closed to retain the lysed solution within the upper chamber 18. After the solution is thoroughly minced, the valve 46 is opened to allow the lysed solution to flow through a filter 48 that is configured to filter particulate matter from the lysed solution formed in the upper chamber 18. After flowing through the filter 48, the filtered solution flows into the lower chamber 22 of the DNA extraction tube.

The lower chamber 22 includes a binding material 50 that binds to the DNA of the filtered solution. In the exemplary embodiment, the binding material 50 includes silica beads 50a that bind to the DNA in the filtered solution. In addition to silica beads, other DNA binding reagents may be employed including silica gel, silica membrane, glass fiber, diatomaceous earth, ion-exchange resin, iron oxide particles or ligand exchange resins. DNA molecules bind to silica, glass, and diatomaceous earth in the presence of high salt concentrations (e.g. Sodium Iodide or Guanidinium Hydrochloride) and low pH. After washing in an ethanol containing high salt buffer, the DNA can be eluted from the silica, glass or diatomaceous earth in a high pH (e.g. pH=8 to 8.5) buffer with low salt concentrations. Ion exchange resins and iron-oxide take advantage of charge-charge interactions for binding DNA. For example, diethylaminoethanol (DEAE) has a positive charge under low salt and low pH conditions and will bind to the negative charge on the DNA phosphate backbone. DNA can be eluted from DEAE under high salt and high pH conditions. The resin or support for ion exchangers can consist of silica, cellulose, dextran or agarose. Iron oxide has the added advantage of magnetism to facilitate collection and concentration of the DNA bound iron-oxide particles.

The lower chamber 22 also includes an inlet 52 for channeling fluids into the lower chamber 22. The inlet 52 is fluidly coupled to an elution tank 54 and a wash buffer tank 56. A valve 58 is positioned within a port 60 coupled to each tank to allow selective flow of either a wash buffer or a hot elution buffer into the lower chamber 22. After the DNA has bound to the binding material 50, a wash solution is channeled through the inlet 52 into the lower chamber 22 to wash the binding material. A waste valve 62 is then opened to channel waste from the lower chamber 22 to a waste chamber 64 that is fluidly coupled to the lower chamber 22 via a waste port 66. The elution buffer is then channeled into the lower chamber 22 to form an eluate in the lower chamber 22. The eluate is channeled from the DNA extraction tube 12 through an elution outlet 68 via an elution valve 70.

More specifically, during extraction of the DNA, the DNA in the filtered solution binds to the silica beads 50a forming a bead slurry. The bead slurry is then mixed by fluid flow or vibration for an incubation period of approximately five minutes. After the incubation period, the solution is drained through the waste port 66 leaving the silica beads 50a having DNA bound thereto. The beads 50a are then washed with the wash solution. For example, the beads 50a may undergo two washes for approximately five minutes each. The wash solution is then drained off and the hot elution buffer having a temperature of approximately 60 degrees Celsius is added to the lower chamber and mixed with the silica beads 50a for approximately five minutes to form the eluate. The eluate is drained through the elution outlet 68 to the chip 14. In the exemplary embodiment, at least one of circuitry, micropumps, microvalves, and/or microheaters/coolers are used to move the eluate from the extraction tube 12 to the chip 14 and/or to maintain a uniform temperature of the eluate when moving the eluate.

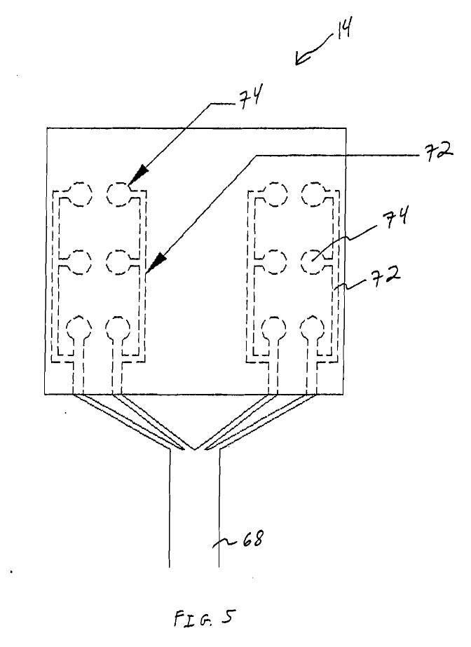

As shown in FIG. 5, the chip 14 includes a plurality of microchannels 72 in fluid communication with a plurality of reaction chambers 74 etched within the chip 14. The eluate is channeled via the microchannels 72 to the reaction chambers 74 where a polymerase chain reaction takes place (PCR) to amplify the DNA in the eluate for detection. Specifically, the reaction chambers 74 include reagents, for example lyophilized nucleotides, DNA polymerase, pathogen specific primers, fluorescent labeling molecules such as SYBR green, and/or reagents specific to a type of PCR that are disposed to create a PCR reaction that specifically amplifies a portion of the DNA on the chip 14. It will be appreciated by one of ordinary skill in the art that the number, size, and orientation of the microchannels 72 and reaction chambers 74 may vary depending on the application of the chip 14. Specifically, the chip 14 is configured to be customized for the detection of various microorganisms. The number, size, and orientation of the microchannels 72 and the reaction chambers 74 may vary based on the particular microorganism sought to be detected. Moreover, in one embodiment, the chip 14 may be configured to detect multiple microorganisms through a single analysis. For example, the meat processing industry is concerned mainly with Escherichia coli, Listeria monocytogenes and Salmonella species. Customized cards for detecting these pathogens could be developed. Processors of fresh fruits and vegetables focus on bacteria such as E. coli, Salmonella, Vibrio cholera, and Shigella; protozoa such as Cryptosporidium parvum, Toxoplasma gondii, Giardia lamblia, Cyclospora cayetanensis; and viruses such as Norwalk and Hepatitis A. For this food processor, custom bacterial, protozoan and viral cards or various combinations could be developed. The dairy industry is concerned with bacterial species from the following genera: Psuedomonas, Bacillus, Clostridium, Corynebacterium, Arthrobacter, Lactobacillus, Microbacterium, Micrococcus, Streptococcus, Listeria, Escherichia, Yersenia, Salmonella, and Campylobacter.

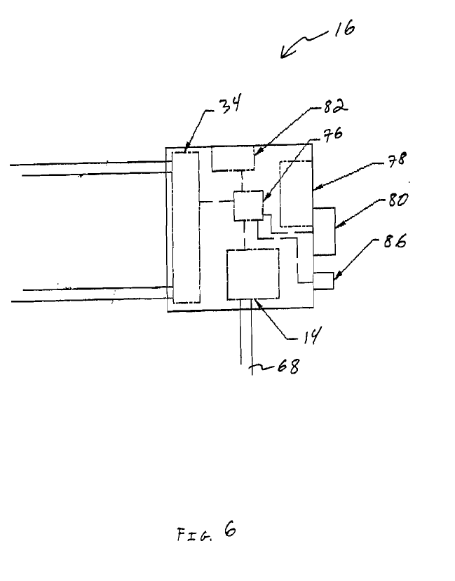

The chip 14 is configured to be inserted into the portable control unit 16, shown in FIG. 6, for detection of microorganisms in the sample through an analysis of the sample's DNA. DNA analysis involves amplification of a portion of a specific pathogen's DNA and labeling the amplified DNA with a tag such a fluorescent tag. Specificity of amplification is achieved with the primers in the PCR reaction mix. Labeling of the amplified DNA may occur during the amplification process if DNA binding fluorescent dyes such as SYBR Green are included in the PCR reaction mix. Alternatively, the primers themselves may be labeled. Cyanine Dyes other than SYBR Green may be used as well as other classes of fluorescent dyes including acridine dyes, fluorone dyes, oxazine dyes, phenanthridine dyes, or Rhodamine dyes. In addition, other classes of labeling molecules may be used including colorometric, bioluminescent or chemiluminescent molecules, semiconductor quantum dots, and lanthide doped compounds or nanoparticles. As an example, fluorescence intensity from labeled amplified DNA can be continuously monitored during the PCR reaction. Fluorescence monitoring requires a light source 100 to excite the fluorophore, filters 104 or monochromators to reduce background from radiation of unwanted wavelengths, a lens 110, a detector 102 of the fluorescence that also converts the light signal to an electrical signal and a data acquisition card for digital readout. In addition, mirrors 106 may need to be incorporated to direct the path of light or alternatively, fiberoptic cables 108 may be used. The excitation energy may be supplied by lamps, light emitting diodes (LED) or lasers. Examples of lamps include tungsten halogen, quartz tungsten halogen, xenon arc and mercury vapor. After excitation, the fluorophore releases energy at a longer wavelength and this energy is detected by the photodetector. Photodetectors include charge coupled devices (CCD), photodiodes, photomultipliers, metal-semiconductor-metal (MSM), phototransistors, photoresistors, and pyroelectric photodetectors. Thus, as the pathogen specific DNA fragment is amplified, more fluorescent dye is bound and excited leading to more signal reaching the photodetector and data acquisition card. In the exemplary embodiment, software upgrades and pathogen specific information are downloadable to the microprocessor 76 to facilitate analysis of the most up to date pathogens and microorganisms.

The control unit 16 includes a digital display 78 that shows the results of the DNA analysis and a built-in battery unit/power supply 80 that allows mobility of the unit 16. In one embodiment, the control unit 16 further includes a global positioning satellite unit 82 to facilitate tracking detected plant pathogens and microorganisms globally and/or to facilitate real-time reporting of bioterrorism threats. The control unit 16 may also include file transfer software that transfers information from the microprocessor 76 to a centralized database 84, as shown in FIG. 1, where the global position of plant pathogens and bioterrorism threats are stored. In the exemplary embodiment, an input/output port 86 is provided to connect the control unit to a personal computer.

Accordingly, the present invention provides a portable microorganism detection device 10 that is capable of detecting microorganisms on food items, crops, or seeds at a point of use of the food item, crop or seed. Specifically, the device 10 is operable at the crop site/production site and provides immediate analysis of pathogens and bioterrorism agents. The present invention further provides a device 10 capable of tracking and reporting pathogen and bioterrorism threats in real-time.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1) A device for detecting a microorganism in a sample, said device comprising:
a DNA extraction tube for extracting an eluate from the sample, said DNA extraction tube comprising an upper chamber having at least one mincing disk to mince the sample, and a lower chamber having a binding material configured to bind to the DNA of the sample, wherein said upper and lower chambers are separated by a filter to capture particulate matter from the sample;
a chip configured to retain the eluate, said chip comprising a reaction chamber disposed for a polymerase chain reaction to amplify DNA in the eluate for detection; and
a control unit configured to pressurize the sample in said DNA extraction tube to facilitate extracting the eluate, said control unit further configured to receive said chip and analyze the amplified DNA on said chip.

2) A device in accordance with claim 1, wherein said upper chamber of said DNA extraction tube comprises a plurality of consecutive mincing disks, wherein at least one of said disks has an incrementally smaller pore size relative to the other said disks.

3) A device in accordance with claim 2, wherein said pores of each said mincing disk comprise serrated edges to facilitate mincing the sample.

4) A device in accordance with claim 1, wherein said lower chamber of said DNA extraction tube comprises an inlet and outlet disposed to channel fluids through said lower chamber.

5) A device in accordance with claim 4, wherein a wash solution is channeled from said inlet to wash the binding material in said lower chamber.

6) A device in accordance with claim 4, wherein an elution buffer is channeled from said inlet to form the eluate in said lower chamber.

7) A device in accordance with claim 6, wherein the eluate is channeled through said outlet of said lower chamber onto said chip.

8) A device in accordance with claim 1, wherein said chip further comprises reagents in said reaction chamber to facilitate the polymerase chain reaction.

9) A device in accordance with claim 8, wherein said reagents include at least one of lyophilized nucleotides, DNA polymerase, pathogen specific primers, and fluorescent labeling molecules.

10) A device in accordance with claim 1, wherein said upper chamber of said DNA extraction tube comprises at least one high pressure port to pressurize the sample in said upper chamber.

11) A device in accordance with claim 1, wherein said control unit further comprises a global positioning unit to facilitate at least one of tracking plant pathogens and reporting bioterrorism threats.

12) A device in accordance with claim 1, wherein said control unit further comprises software and hardware for wireless data transfer to and from the control unit.

13) A device in accordance with claim 1, wherein said control unit further comprises a display screen to display the results of the DNA analysis.

14) A DNA extraction tube for extracting DNA from a sample, said DNA extraction tube comprising:
an upper chamber for receiving the sample, said upper chamber having at least one mincing disk to mince the sample, wherein said at least one mincing disk comprises at least one pore lined with serrated edges to facilitate mincing the sample, said upper chamber configured to be pressurized to force the sample through said mincing disks;
a filter to filter particulate matter from the sample; and
a lower chamber configured to receive a filtered sample from said filter, said lower chamber having a binding material configured to bind to the DNA of the sample, said lower chamber further comprising an inlet for channeling fluids to said lower chamber and an outlet for channeling fluids from said lower chamber.

15) A DNA extraction tube in accordance with claim 14, wherein said upper chamber comprises a plurality of consecutive mincing disks, wherein at least one of said disk has an incrementally smaller pore size than the other said disks.

16) A DNA extraction tube in accordance with claim 14, wherein said binding material in said lower chamber includes silica beads.

17) A DNA extraction tube in accordance with claim 14, wherein a wash solution is channeled from said inlet to said lower chamber to wash said binding material in said lower chamber.

18) A DNA extraction tube in accordance with claim 4, wherein an elution buffer is channeled from said inlet to form an eluate in said lower chamber.

19) A chip for analyzing DNA in a sample, said chip comprising a reaction chamber disposed for a polymerase chain reaction to amplify DNA in the sample for detection, said chip sized for insertion into a control unit that analyzes the DNA after amplification.

20) A chip in accordance with claim 19, wherein said chip further comprises reagents in said reaction chamber for amplification of the DNA, wherein said reagents include at least one of lyophilized nucleotides, DNA polymerase, pathogen specific primers, and fluorescent labeling molecules.

21) A chip in accordance with claim 18, wherein the DNA is analyzed to detect at least one of food, plant pathogens and/or bioterrorism molecules.
